(19) United States
(12) Patent Application Publication
(10) Pub. No.: US 2005/0268904 A1
(43) Pub. Date: Dec. 8, 2005

(54) Device for delivering medication

(76) Inventor: Craig Corey, Davidson, NC (US)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/862,491

(22) Filed: Jun. 7, 2004

(51) Int. Cl. A61M 11/00; B05B 3/02

(52) U.S. Cl. 128/200.14; 128/200.17; 128/200.18

(57) ABSTRACT

There is provided a device to augment the delivery of medication to a patient in aerosolized or gaseous form. The device includes a telephone handle shaped device having a gripping portion, an earpiece at one end of the gripping portion and a mouthpiece having a medical supply located at the other end of the gripping portion. The medical supply comprising a medication supply inlet, a medication supply outlet and a rotating connector seated in the mouthpiece between the medication supply inlet and the medication supply outlet. A nebulizer is attached to the rotating connector so that when the device is used, the medication is delivered through the medical supply outlet in the mouthpiece adjacent the patient's mouth. The earpiece may include means to generate music or the like. The delivery device is especially useful for use with small children and infants.

### BACKGROUND OF THE INVENTION

1. Field of the Invention

The present invention relates to an improved a device for the delivery of medication. More particularly, the present invention relates to an improved hand held device to augment the delivery of medication in aerosolized or gaseous form.

2. Description of the Prior Art

Millions of patients worldwide are affected with acute and chronic respiratory diseases, such as asthma, croup, cystic fibrosis (CF), respiratory syncytial virus (RSV), pneumonia, bronchopulmonary dysplasia (BPD) and bronchitis. These respiratory diseases frequently require a regular program of medication which is often given on a daily basis in aerosolized form. Medications frequently utilized in the treatment of asthma include bronchodilators, such as albuterol and anti-inflammatory agents, such as cromolyn sodium and leukotriene inhibitors. Antibiotics, such as pentamidine, are nebulized in the treatment of AIDS pneumonia. Medications used in the treatment of CF may be nebulized. In addition, there is sometimes the need to provide oxygen to a patient. Although treatment for certain respiratory diseases is practiced on a regular basis there are often unexpected attacks among both older and younger patients.

Respiratory attacks provide particular anxiety when they occur in infants or small children below the age of six or so. The presently used delivery devices often require a mask to be placed over or held in close proximity to the child's face to be effective. These small children are quite frequently anxious and uncooperative at the time the medication is needed due to their illness and unfamiliar surroundings. The presently used medication delivery devices tend to increase the anxiety, crying and combative behavior of the child. This not only makes the delivery of the medication difficult, it stresses the parents and care givers.

One such delivery device for delivering medication to a child is described in U.S. Pat. No. 5,803,063 to Corey which discloses a telephone handle shaped device having a gripping portion, an ear piece at one end and a mouthpiece at the other end. The mouth piece includes a nebulizer for supplying medication coupled to the mouthpiece of the handle. This delivery device, while quite efficient, is not easily rotated to positions to accommodate a patient sitting or laying on a bed.

### SUMMARY OF THE INVENTION

It is an object of the present invention to provide a device to augment the aerosolized delivery of medication to a patient that has a rotating connector in the mouthpiece to cause the nebulizer to swivel.

Another object of the present invention is to provide a hand held device to augment the delivery of medication in aerosolized or gaseous form to an infant or small child when the patient is lying flat in a bed or other position such as sitting.

The present invention provides a device to augment the delivery of medication, such as asthma drugs, antibiotics, oxygen or the like in aerosolized or gaseous form to a patient. The delivery device includes a means for supplying medication in combination with a supply device in the shape of a telephone handle having a gripping portion and an earpiece at one end of the gripping portion and a telephone mouthpiece at the other end of the gripping portion. The mouthpiece is attached to the means for providing medication. The medication supply means is preferably a conventional nebulizer. The mouthpiece has therein a receptacle, for example, a 17 mm opening, designed to accept a conventional nebulizer unit or extension thereof without adaptation. Other nebulizer units may have slightly variable sizes unique to their products. The insertion opening in the mouthpiece may be of flexible material which would allow for some variation in size of the inner end of the rotating connector. The nebulizer may be attached by inserting the stem of the nebulizer unit into the outer end of the rotating connector, which is held in place by friction or other means.

The nebulizer includes a rotating connector which allows the telephone handle to be used in almost any position, including lying flat in a bed or with a patient lying on his or her side in the bed. The rotating connector is a passive mechanism requires no adjustment by the patient or caregiver. The pull of the connection tubing provides the force to keep the swivel in the desired position.

In a preferred embodiment of the invention, the earpiece of the handle includes a musical device which plays while the patient is receiving medication and results in a soothing effect.

In yet another embodiment, there is provided an extender for use with a mask. This extender fits into the bowl of the mouthpiece portion of the device. A mask may then be attached to provide for higher oxygen concentrations if necessary.

The aerosolized medicine flows freely to the patient's mouth and nose through the open mouthpiece. The medication delivery device is especially useful for treating small children and infants.

### BRIEF DESCRIPTION OF THE DRAWINGS

The foregoing and other objects, features and advantages of the invention, and the manners in which the same are accomplished, will become apparent from the following detailed description of the invention taken in conjunction with the accompanying drawings which illustrate preferred and exemplary embodiments, wherein:

FIG. 1 is an environmental view of a small child in an upright position using the medication delivery device of the present invention;

FIG. 2 is a perspective view of a preferred embodiment of the medication delivery device of the present invention illustrating the rotating connector;

FIG. 3 is a partial view of another embodiment of the medical supply portion of the delivery device of the present invention showing the rotating connector and the use of a mask attached to the extender inserted into the handle mouthpiece;

FIG. 4 is a partial cutaway view showing the rotating connector seated in the telephone handle taken along line 4-4 of FIG. 2;

FIG. 5 illustrates a small child being administered medication using the medication delivery device of the present invention in a lying position; and

FIG. 6 shows another embodiment of the medication delivery device of the present invention designed for long-term use, being attached to a source of music.

### DETAILED DESCRIPTION OF THE PREFERRED EMBODIMENTS

The present invention will now be described more fully hereinafter with reference to the accompanying drawings, in which a preferred embodiment of the invention is shown. This invention may, however, be embodied in many different forms and should not be construed as limited to the embodiments set forth herein; rather, this embodiment is provided so that this disclosure will be thorough and complete and will fully convey the scope of the invention to those skilled in the art.

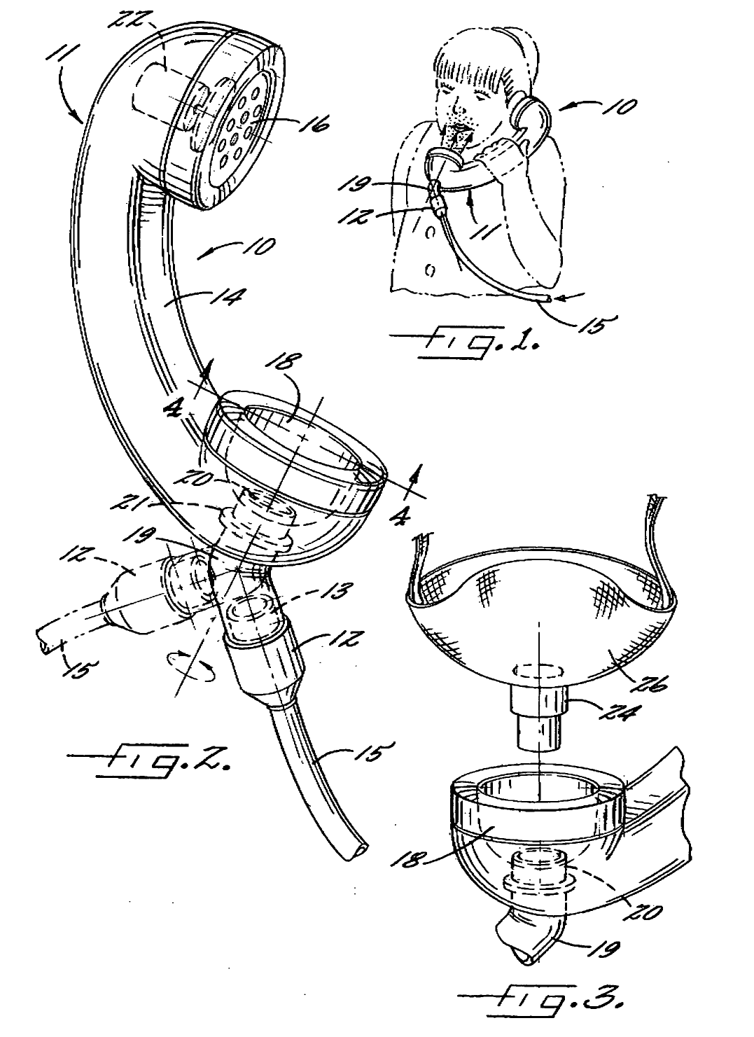

Referring now to FIG. 1, there is shown an environmental view of a small child using the medication delivery device 10 of the present invention sitting or standing in an upright position. As shown more clearly in FIG. 2, there is provided a delivery device 10 that includes a handle in the shape of a telephone handle 11 having a gripping portion 14, an earpiece 16 at one end of the gripping portion 14 and a mouthpiece 18 at the other end of the gripping portion 14 and a medication supply 12, such as a nebulizer, for supplying medication in aerosolized or gaseous form. As used herein the term medication includes, bronchodilators, such as albuterol; anti-inflammatory agents, such as cromolyn sodium and leukotriene inhibitors; antibiotics; such as pentamidine; medications used in the treatment of CF; and where applicable, oxygen. The medication supply 12 is connected to the mouthpiece 18. The mouthpiece 18 has therein a receptacle, for example, a 17 mm opening, designed to accept a conventional nebulizer unit or extension thereof without adaptation.

The rotating connector 19 allows the delivery device 10 to be used in almost any position, as will be further described hereafter. The medication supply 12 may be a conventional nebulizer having a medication supply inlet which is attached to a supply line 15 from a nebulizer pressure source and an outlet, such as an “acorn” nebulizer from Nellcor Puritan Bennett. The external mouthpiece receptacle 20 is an opening of variable size designed to accept existing nebulizer units without adaptation. The inner end of rotating connector 19 extends through mouthpiece receptacle 20. The attachment of the medication supply means 12 to mouthpiece 18 is accomplished by inserting the stem 13 of the nebulizer unit 12 into the outer end of the rotating connector 19. The device will enhance the delivery of medication because the telephone is a play toy to every child. It is a familiar object generally associated with the child's regular environment. It is not a threatening, unknown object thrust over the child's mouth.

Optionally, the earpiece 16 of the telephone-shaped handle 11 may include a means 22 to generate music or the like. The means 22 may be a computer chip having prerecorded music or a story such as those found in, for example, greeting cards or a speaker attached to an external source. In this embodiment the music means 22 will further enhance the child's comfort with the situation ensuring appropriate delivery of needed medications and easing the stress for all involved.

As shown in FIG. 1 the dimensions of the telephone-shaped handle 11 are appropriate to fit a child's smaller ear to mouth distance. With placement of the telephone to the child's ear, the mouth of the nebulizer to the child's mouth and nose occurs without further maneuvers. The telephone-shaped handle may be made of plastic, and stay with the child during his or her stay in the office, emergency room or the like. The aerosolized medicine flows freely to the child's facial area through the open mouthpiece 18. This version of the invention is cleanable to allow for re-use.

In another embodiment, that shown in FIG. 3, there is provided a mouthpiece 18 having an extender 24 for use with a mask 26. The extender 24 extends through the opening in mouthpiece 18 to connect to the rotating connection 19. The receptacle on the base of the mouthpiece is the insertion point for the nebulizing device. In this embodiment, a simple adaptor will allow an oxygen mask to be placed in the mouthpiece to fit over the face if required, still delivering the needed medication or oxygen with the benefit of the phone.

The above-described device to augment delivery of mediation to small children may be produced so as to be disposable, especially desirable with devices used in hospitals, clinics, offices or ambulances for limited use and subsequent disposal. However, it is also desirable that the delivery devices of this invention may be produced for multiple-use application, particularly at home, where the handle 11 would be made of injection molded plastics suitable for home cleaning.

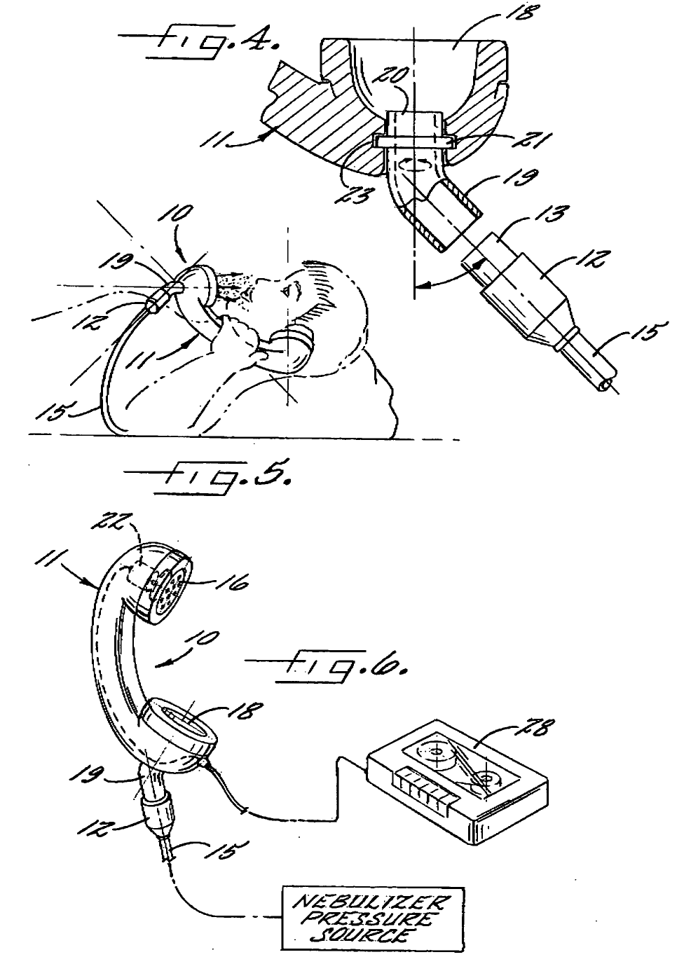

In FIG. 4, there is shown a cutaway of the rotating connector 19 seated in the telephone handle. The rotating connector 19 is a passive mechanism requiring no adjustment by the patient or care giver. The pull of the connection tube provides the force to keep the “acorn” in the desired position. The rotating connector 19 rotates freely through 360° allowing use of the telephone handle by a righthanded or lefthanded person without adjustment. In addition, the device because it rotates freely through 360° easily allows for changes in position of the patient from sitting to lying, as shown in FIG. 5, without adjustment of the device. As shown in FIG. 4, the rotating connector 19 is disposed at an angle which allows for use of the nebulizer at angles of up to 135° allowing continuous delivery of medication while lying flat in the bed, again as shown in FIG. 5. The rotating connector 19 preferably is disposed at about a 45° to the angle of use of the nebulizer without any adjustments or additions of tubing or connectors, i.e., the rotating connector outlet is disposed at about a 45° angle from the nebulizer. The mouthpiece 18 has an indented portion 23 of appropriate size to accommodate the lip 21 of the rotating connector around its interior. The rotating connector is seated in the telephone handle mouthpiece as a component of the medication device. Thus, there are no additional parts to attach for use.

As noted, this passive mechanism automatically adjusts to the position of the patient. The design of the rotating connector 19 directs the medication flow to the mouth and nose area without any adjustment no matter the angle of the nebulizer.

In FIG. 6, there is shown another embodiment of the medication delivery device 10 of the present invention which is particularly useful for home or extended use. As shown in FIG. 5, an audio system 28 is attached to the mouthpiece 18 of the telephone-shaped handle 11 and the parents or care givers can play age-appropriate tapes of music, songs or stories to calm the child while the medication is being delivered.

Many modifications and other embodiments of the invention will come to mind to one skilled in the art to which this invention pertains having the benefit of the teachings present in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are use in a generic and descriptive sense only and not for purposes of limitation.

1. A hand held device to augment the delivery of medication in aerosolized or gaseous form to a child during an asthma attack comprising:
a telephone handle shaped device having a gripping portion, an earpiece at one end of said gripping portion and a mouthpiece having a medical supply located at the other end of said gripping portion, said medical supply comprising a medication supply inlet, a medication supply outlet and a rotating connector seated in said mouthpiece between said medication supply inlet and said medication supply outlet capable of rotating while said device is being used; and a nebulizer attached to said rotating connector so that when said device is used, said medication is delivered through said medical supply outlet adjacent said patient's mouth.

2. The device according to claim 1 wherein said rotating connector seated in said mouth piece is capable of rotating 360°.

3. The device according to claim 1 wherein said medication supply outlet is disposed at about a 45° angle from said nebulizer.

4. The device according to claim 1 wherein said means for augmenting the delivery of said medication includes an adapter allowing attachment of a mask to the mouthpiece of the telephone-shaped handle, enabling said mask to fit over said child's face.

5. The device according to claim 1 wherein said earpiece includes a device for providing music.

6. The device according to claim 1 wherein said telephone handle shaped device is plastic.
